SEQUENCE LISTING (31 sequences)

SEQ ID NO: 1 - DNA, 29 bp, Artificial
  Synthetic Construct
cgactcactatagggttttttttttttvn

SEQ ID NO: 2 - RNA, 22 nt, Homo sapiens
uagcuuaucagacugauguuga

SEQ ID NO: 3 - DNA, 27 bp, Artificial
  Synthetic Construct
aaaaaaaatagcttatcagactgatgt

SEQ ID NO: 4 - DNA, 29 bp, Artificial
  Synthetic Construct
cgactcactatagggttttttttttttca

SEQ ID NO: 5 - RNA, 22 nt, Arabidopsis thaliana
ugaagcugccagcaugaucugg

SEQ ID NO: 6 - DNA, 27 bp, Artificial
  Synthetic Construct
aaaaaaaaatgaagctgccagcatgat

SEQ ID NO: 7 - DNA, 29 bp, Artificial
  Synthetic Construct
cgactcactatagggttttttttttttcc

SEQ ID NO: 8 - DNA, 45 bp, Homo sapiens
ctgcgcaaggatgacacgcaaattcgtgaagcgttccatattttt

SEQ ID NO: 9 - DNA, 20 bp, Artificial
  Synthetic Construct
ctgcgcaaggatgacacgca

SEQ ID NO: 10 - DNA, 29 bp, Artificial
  Synthetic Construct
cgactcactatagggttttttttttttaa

SEQ ID NO: 11 - RNA, 22 nt, Homo sapiens
uggagugugacaaugguguuug

SEQ ID NO: 12 - DNA, 28 bp, Artificial
  Synthetic Construct
aaaaaaaatggagtgtgacaatggtgtt

SEQ ID NO: 13 - DNA, 24 bp, Homo sapiens
ggaataccgggtgctgtaggcttt

SEQ ID NO: 14 - DNA, 28 bp, Artificial
  Synthetic Construct
aaaaaaaaaggaataccgggtgctgtag

SEQ ID NO: 15 - RNA, 22 nt, Populus trichocarpa
uagcuuaucagacugauguuga

SEQ ID NO: 16 - DNA, 28 bp, Artificial
  Synthetic Construct
aaaaaaaaatgcactgcctcttccctgg

SEQ ID NO: 17 - DNA, 29 bp, Artificial
  Synthetic Construct
cgactcactatagggttttttttttttgc

SEQ ID NO: 18 - RNA, 21 nt, Populus trichocarpa
ucggaccaggcuucauucccc

SEQ ID NO: 19 - DNA, 26 bp, Artificial
  Synthetic Construct
aaaaaaaatcggaccaggcttcattc

SEQ ID NO: 20 - DNA, 29 bp, Artificial
  Synthetic Construct
cgactcactatagggttttttttttttgg

SEQ ID NO: 21 - RNA, 21 nt, Populus trichocarpa
ucggaccaggcuucauuccuu

SEQ ID NO: 22 - DNA, 26 bp, Artificial
  Synthetic Construct
aaaaaaaatcggaccaggcttcattc

SEQ ID NO: 23 - RNA, 21 nt, Populus trichocarpa
ugaagcugccagcaugaucua

SEQ ID NO: 24 - DNA, 27 bp, Artificial
  Synthetic Construct
aaaaaaaaatgaagctgccagcatgat

SEQ ID NO: 25 - DNA, 29 bp, Artificial
  Synthetic Construct
cgactcactatagggttttttttttttag

SEQ ID NO: 26 - RNA, 21 nt, Populus trichocarpa
ugaagcugccagcaugaucuu

SEQ ID NO: 27 - DNA, 27 bp, Artificial
  Synthetic Construct
aaaaaaaaatgaagctgccagcatgat

SEQ ID NO: 28 - RNA, 21 nt, Populus trichocarpa
ugaagcugccagcaugaucug

SEQ ID NO: 29 - DNA, 27 bp, Artificial
  Synthetic Construct
aaaaaaaaatgaagctgccagcatgat

SEQ ID NO: 30 - RNA, 24 nt, Populus trichocarpa
ggcacgucugccugggugucacgc

SEQ ID NO: 31 - DNA, 27 bp, Artificial
  Synthetic Construct
aaaaaaaaacgtctgcctgggtgtcac

(19) United States
(12) Patent Application Publication
(10) Pub. No.: US 2009/0220969 A1
(43) Pub. Date: Sep. 3, 2009

(54) Identifying and quantifying small RNAs

(75) Inventors: Vincent Chiang, Cary, NC (US); Rui Shi, Raleigh, NC (US)

Correspondence Address: THE ECLIPSE GROUP LLP, 10605 BALBOA BLVD., SUITE 300, GRANADA HILLS, CA, 91344, US

(73) Assignee: North Carolina State University, Raleigh, NC (US)

(21) Appl. No.: 12/231,525

(22) Filed: Sep. 3, 2008

Related U.S. Application Data
(60) Provisional application No. 60/995,770, filed on Sep. 28, 2007.

(51) Int. Cl. C12Q 1/68; C12P 19/34; C12N 9/12

(52) U.S. Cl. 435/6; 435/91.2; 435/194

(57) ABSTRACT

One-step RT-PCR methods, compositions and kits for the detection and quantification of small RNAs in a sample are disclosed. The one-step RT-PCR approach involves polyadenylation of a small RNA followed by reverse transcription with a first primer containing a poly(T) sequence and at least two 3′ nucleotides complementary to the 3′ terminal end nucleotides of the small RNA, to produce a cDNA. This may be followed by PCR amplification using the same first primer as the revere primer and a second, forward primer in which a portion of its sequence is complementary to the 3′ terminal end of the cDNA. This may be then followed by detection and/or quantification of the amplified product.

### FIELD

The present teachings relate to methods, compositions and kits for amplifying, identifying, and quantifying small RNA molecules (small RNAs).

### INTRODUCTION

Small RNAs of about 19-30 nucleotides (nt) in length, play an important role in a remarkable range of biological pathways. Recent studies have demonstrated that small RNAs can act as regulators that control plant and animal gene expression via gene silencing mechanisms (for review, see Zamore & Haley, 2005, Science 309: 1514-1524; Kim 2005, Mol. Cells 19:1-15). The importance of this gene silencing mechanism has become apparent in the observation that, one class of small RNAs, the microRNA (miRNA), may regulate at least one-third of all human genes (Lewis et al. 2005, Cell 120:15-20).

Several distinct classes of small RNAs have been identified. These include, for example, microRNA (miRNA: Lee et al. 1993, Cell 75:843-854; Reinhart et al., 2000, Nature 403:901-906); small interfering RNA (siRNA: Hamilton et al. 1999, Science 286:950-952; Hammond et al. Nature 404:293-296); repeat-associated small interfering RNA (rasiRNA: Reinhart and Bartel 2002, Science 297:183; Volpe et al., 2002, Science 297: 1833-1837) and the newly discovered, PIWI-interacting RNA (piRNA: Grivna et al. 2006, Genes Dev. 20:1709-1714).

In order to study the small RNAs, several approaches for detecting the small RNAs have been utilized. These including, for example, Northern blotting hybridization combined with PAGE separation (Reinhart et al., 2000, Nature 403: 901-906; Lagos-Quintana et al. 2001, Science 294:853-858; Lee and Ambros, 2001, Science 294:862-864) primer extension (Zeng and Cullen 2003, RNA 9: 112-123) and RNase protection (see, for example, RPA III™ Ribonuclease Protection Assay Kit, Ambion, An Applied Biosystems Business, Austin, Tex.).

RT-PCR assay approaches for detecting small RNAs have also been considered because such approaches could potentially provide a higher throughput and greater sensitivity than the assays described above. Nevertheless, the small size of small RNAs has presented a significant challenge for the development of an RT-PCR assay for small RNAs. However, various approaches to lengthen the sequence and facilitate an RT-PCR assay have been developed. These approaches have generally involved the use of an adapter containing an arbitrary sequence of nucleotides to extend the length of the small RNA or the corresponding cDNA to permit PCR amplification. For example, extension of the sequence has been achieved by reverse transcription using a stem-loop adapter that has a 3′ end complementary to the miRNA (Chen et al., 2005, Nucleic Acids Res. 33:e179, 2005); by reverse transcription with a linear adapter that is used in conjunction with short primers containing LNA bases (Raymond et al., 2005, RNA 11: 1737-1744, 2005); and by ligation of a linear adapter to the miRNA using T4 RNase Ligase (Grad et al., 2003, Molecular Cell 11: 1253-1263, 2003). Another RNA-extension method lengthened the miRNA by polyadenylation and reverse transcription with a poly(T) adapter (Shi and Chiang, 2005, BioTechniques 39: 519-525). All of these methods were incorporated into two-step RT-PCR methods.

In general, two-step RT-PCR methods perform the RT and PCR steps either in a first and then a second tube into which the sample is transferred for successive reactions or in a single tube in which different reagents are added for the RT and PCR steps. One-step RT-PCR methods perform cDNA synthesis in the RT step and amplification in the PCR step in a single tube using the same buffer and site-specific primers. As a result, there are certain advantages of one-step RT-PCT over two step RT-PCR methods. For example, assay time is minimized because fewer pipetting steps are required; the risk of contamination is reduced because no transfers are required and there is no need to open the reaction tube to add reagents; and the sensitivity of the assay is also improved (Wacker and Godard, 2005, Analysis of One-Step and Two-Step Real-Time RT-PCR Using SuperScript III, J. Biomol. Tech. 16:266-271). These advantages would make it desirable in certain instances to use a one-step RT-PCR method for the detection and quantification of small RNAs. Thus, there is a need for a one-step RT-PCR method for detecting and quantifying small RNAs.

### SUMMARY

Accordingly, the present teachings describe, in various embodiments, one-step RT-PCR methods, compositions and kits for the detection and quantification of small RNAs. The RT-PCR approach involves polyadenylation of the small RNA followed by reverse transcription with a first primer containing a poly(T) sequence. This is followed by PCR amplification using the same first primer as the reverse primer and a second, forward primer in which a portion of its sequence is complementary to the 3′ terminal end of the cDNA. This is then followed by detection and/or quantification of the amplified product.

Thus, in various embodiments, the present teachings provide a method for detecting and/or quantifying a small RNA. The method comprises polyadenylating the small RNA with ATP and a poly(A) polymerase to form a polyadenylated small RNA having a sequence of contiguous A residues. The sequence of contiguous A residues may be 12 or more A residues. Subsequently, the polyadenylated small RNA is reverse transcribed to produce a cDNA in a reaction mixture that includes (i) the polyadenylated small RNA; (ii) a first primer of not more than 40 nucleotides in length having complementarity to at least two 3′ terminal end nucleotides of the small RNA and the sequence of contiguous A residues of the polyadenylated small RNA so as to hybridize therewith and initiate synthesis of a cDNA complementary to the polyadenylated small RNA; (iii) a reverse transcriptase; and (iv) all four deoxyribonucleoside triphosphates. Then, a DNA molecule that includes the cDNA sequence is amplified by a polymerase chain reaction (PCR) in a reaction mixture that includes (i) the cDNA, (ii) the first primer; (iii) a second primer that is sufficiently complementary to the 3′ nucleotides of the cDNA to hybridize with the cDNA and initiate synthesis of an extension product; (iv) a DNA polymerase; and (v) all four deoxyribonucleoside triphosphates. Subsequently, the amplified DNA molecule is detected and/or quantified if present, wherein the presence and/or quantity of the amplified DNA corresponds to the presence and/or quantity, respectively, of the small RNA. In various embodiments, both the reverse transcription and the PCR are performed in a single tube and in the same mixture of reagents.

In other embodiments, the present teachings provide a method of amplifying a polyadenylated small RNA that has a sequence of contiguous A at the 3′ terminal end. The sequence of contiguous A residues may be 12 or more A residues. The method includes reverse transcribing the polyadenylated small RNA to form a cDNA in a reaction mixture that includes (i) the polyadenylated small RNA; (ii) a first primer of not more than 40 nucleotides in length having complementarity to at least two 3′ terminal nucleotides of the small RNA and the sequence of contiguous A residues of the polyadenylated small RNA so as to hybridize therewith and initiate synthesis of a cDNA complementary to the polyadenylated small RNA; (iii) a reverse transcriptase; and (iv) all four deoxyribonucleoside triphosphates. Subsequently, a DNA molecule that includes the cDNA sequence is amplified by pcr in a reaction mixture that includes (i) the cDNA, (ii) the first primer; (iii) a second primer that is sufficiently complementary to the 3′ nucleotides of the cDNA to hybridize with the cDNA and initiate synthesis of an extension product; (iv) a DNA polymerase and (v) all four deoxyribonucleoside triphosphates. In various embodiments, both the reverse transcription and the PCR are performed in a single tube and in the same mixture of reagents.

In still other embodiments, the present teachings provide compositions that form a reaction mixture for performing an RT-PCR method on a polyadenylated small RNA. The reaction mixture includes (a) a sample containing a small RNA that has been polyadenylated to contain a sequence of contiguous A residues at the 3′ terminal end, (b) a first primer of not more than 40 nucleotides in length having complementarity to at least two 3′ terminal nucleotides of the small RNA and the sequence of contiguous A residues of the polyadenylated small RNA so as to hybridize to the polyadenylated small RNA and initiate synthesis of a cDNA complementary to the polyadenylated small RNA, (c) a second primer that is complementary to the 3′ nucleotides of the cDNA so as to hybridize with the cDNA and initiate synthesis of an extension product, (d) a reverse transcriptase, (e) a DNA polymerase and (f) all four deoxyribonucleoside triphosphates. The sequence of contiguous A residues may be 12 or more A residues.

In other embodiments, the present teachings provide a method of amplifying a small RNA that has been polyadenylated to contain sequence of contiguous A residues at the 3′ end. The sequence of contiguous A residues may be 12 or more A residues. The method comprises (a) forming a first reaction complex comprising a first DNA primer of not more than 40 nucleotides in length, hybridized to a portion of the polyadenylated small RNA containing at least two nucleotides that formed the 3′ terminal end of the small RNA prior to polyadenylation and the sequence of contiguous A residues; (b) extending the first DNA primer to form an elongated cDNA molecule complementary to the polyadenylated small RNA; (c) separating the elongated cDNA molecule from the polyadenylated small RNA; (d) forming a second reaction complex comprising a second DNA primer hybridized to the 3′ end of the elongated cDNA molecule; (e) extending the second DNA primer to form a first strand; (f) separating the first strand from the elongated cDNA molecule; (g) forming a third reaction complex comprising the first strand and the first DNA primer; (h) extending the first DNA primer to form a second strand, wherein the first strand is hybridized to the second strand to form a double stranded complex; and (i) amplifying the double stranded complex.

In still other embodiments, the present teachings provide a kit for amplifying and detecting and/or quantifying the presence of a small RNA in a sample. The kit includes a primer set comprising a first primer of not more than 40 nucleotides in length having at least two nucleotides complementary to the 3′ terminal end nucleotides of the small RNA and a sequence of contiguous A residues 3′ to the at least two nucleotides such that the first primer hybridizes to a polyadenylated form of the small RNA. The primer set further comprises a second primer that is sufficiently complementary to the 3′ nucleotides of the cDNA to hybridize with the cDNA and initiate synthesis of an extension product. In some implementations, the kit may also contain a poly(A) polymerase for converting the small RNA into a polyadenylated small RNA prior to amplification. The polyadenylated small RNA formed with the polymerase has a sequence of contiguous A residues at the 3′ terminal end. The sequence of contiguous A residues may be 12 or more A residues. The kits may contain a reverse transcriptase and a DNA polymerase. In some implementations, the kits may also contain a poly(A) polymerase for generating the polyadenylated form of the small RNA.

### BRIEF DESCRIPTION OF THE FIGURES

FIG. 1 is a schematic drawing illustrating a one-step RT-PCR method for small RNAs as applicable to an miRNA in which (A) the miRNA is polyadenylated by E. coli poly(A) polymerase (E-PAP) and ATP; (B) the polyadenylated miRNA is reverse transcribed into a first strand cDNA using an miRNA-specific poly(T) anchor (reverse) primer; and (C) cDNA of the miRNA is amplified using the same miRNA-specific anchor primer and an miRNA-specific forward primer.

FIG. 2 illustrates the validation of amplification of small RNA by an RNA-extension based, one-step RT-PCR method according to an embodiment, showing (A) dissociation curve analysis of one-step PCR products for human miRNA hsa-mir-21, Arabadoposis miRNA Ath-MIR167d and human snRNA U6; (B) detection of one-step PCR products for the same three small RNAs by electrophoresis on 12% denatured PAGE; and (C) the expected size and sequences of PCR products for the same three small RNAs.

FIG. 3 illustrates the dynamic range and sensitivity of a one-step RT-PCR method for small RNAs according to an embodiment, showing (A) an amplification plot of synthetic human hsa-mir-21 over 7 orders of magnitude in which synthetic miRNA input ranged from 6 to 6×10⁷ copies in a 20 μl PCR reaction; (B) a standard curve for hsa-mir-21 in which synthetic miRNA input ranged from 6 to 6×10⁷ copies in a 20 μl PCR reaction; and (C) a standard curve for hsa-mir-21 in which total RNA input ranged from 0.1 pg to 100 ng in a 20 μl PCR reaction.

FIG. 4 illustrates the specificity of a one-step RT-PCR method for small RNAs using perfectly matched and mismatched primer sets for human hsa-mir-21 showing (A) primer set sequences with mismatches enclosed in boxes; and (B) the relative abundance level of PCR products using the perfectly matched primer set and the various mismatched primer sets.

FIG. 5 illustrates the quantification of a polyadenylated miRNA precursor showing (A) the predicted sequence and hairpin structure of human mir-22 miRNA polyadenylated precursor along with primer set sequences; (B) an amplification plot of one-step real time RT-PCR for human mir-22 mature miRNA and its polyadenylated precursor; and (C) a bar-chart representing the relative abundance of mature human mir-22 mature miRNA and its polyadenylated precursor.

FIG. 6 illustrates the quantification of tissue-specific expression of various miRNAs by one-step real-time RT-PCR, by RT-PCR with end-point quantification and by Northern hybridization showing (A) relative tissue-specific miRNA expression in human brain (Br), heart (Hr), kidney (Ki), liver (Li), lung (Lu), skeletal muscle (Sk), spleen (Sp) and thymus (Th) for hsa-miR-21 and hsa-miR-122 and in plant (P. trichocarpa) leaf (L), phloem (P), shoot tip (S) and xylem (X) for ptc-miR-408, ptc-miR-166 and ptc-miR-167 using one-step real-time RT-PCR; (B) end-point detection of PCR amplified reference rRNAs, human 5S rRNA and P. trichocarpa 5.8S rRNA using polyacrylamide gel electrophoresis and ethidium bromide staining; (C) end-point detection of PCR amplified hsa-miR-21, hsa-miR-122, ptc-miR408, and members of the ptc-MIR166 family (types 1 and 2 identified as Seq. 1 and Seq. 2, respectively) and the ptc-MIR167 family (types 1, 2 and 3 identified as Seq. 1, Seq. 2 and Seq. 3, respectively) using polyacrylamide gel electrophoresis and ethidium bromide staining; (D) ethidium bromide stained reference rRNA transcripts separated on polyacrylamide/urea gel as the loading control; and (E) Northern blot analysis of tissue-specific expression patterns of hsa-miR-21, hsa-miR-122, ptc-miR408, ptc-MIR166 and ptc-MIR167.

### DESCRIPTION OF EXEMPLARY EMBODIMENTS

The present teachings describe a one-step RT-PCR approach for detecting and/or quantifying small RNAs.

Various types of small RNAs have been identified and these have been determined to have from about 19 nucleotides up to about 30 or about 31 nucleotides or more. (See for example, Kim, 2006, Genes & Development 20:1993-1997). The term “small RNA” is intended to reference these and other RNA molecules some of which have a length of from about 19 up to about 23 nucleotides while others have a length up to about 30 or 31 nucleotides. Such small RNAs may include miRNAs, siRNAs, rasiRNAs, piRNAs or any other RNA having a length as described above. The small RNAs may also include certain small nuclear RNAs (snRNAs) of not more than about 45 nucleotides in length.

Samples containing small RNAs may include animal cells and/or tissue, plant tissue, bacteria, or yeast or any preparation derived from such sources as well as synthetic sequences. In general, total RNAs may be initially purified from such samples using any suitable method that insures efficient recovery of small RNAs. Total RNA purification methods are well known in the art and commercially available kits for RNA purification provide reagents and methodology for lysis of the cells or tissue followed by separation of the RNA from the lysed sample. (See for example, TRIzol® Plus RNA Purification System, Invitrogen Carlsbad, Calif.).

Thus, the present teachings provide methods for detecting and/or quantifying a small RNA. The term “detecting and/or quantifying” is intended to mean either or both of detecting and quantifying the small RNA. Detecting includes determining whether the small RNA is present or absent in the original sample at a level that can be measured by the assay system and quantifying includes determining the amount of the small RNA present in the original sample in relative or absolute values.

The term “hybridize” or “hybridization” as used in connection with the RT-PCR methods of the present teachings, refers to the process of annealing complementary nucleic acid strands by forming hydrogen bonds between nucleotide bases on the complementary nucleic acid strands. Hybridization, and the strength of the association between the nucleic acids, may be impacted by such factors as the degree of complementarity between the hybridizing nucleic acids, the stringency of the conditions involved, the melting temperature, Tₘ of the formed hybrid, and the G:C ratio within the nucleic acids. The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acids that have a high frequency of complementary base sequences. With “weak” or “low” stringency conditions nucleic acids the frequency of complementary sequences is usually less, so that nucleic acids with differing sequences can be detected and/or isolated.

Thus, in various embodiments, the present teachings provide methods for amplifying, detecting and/or quantifying a small RNA. The methods may include polyadenylating the small RNA with ATP and a poly(A) polymerase. Subsequently, the polyadenylated small RNA is reverse transcribed to produce a cDNA, in a reaction mixture that includes (i) the polyadenylated small RNA; (ii) a first primer having sufficient complementarity to the polyadenylated small RNA to hybridize therewith and initiate synthesis of a cDNA; (iii) a reverse transcriptase; and (iv) all four deoxyribonucleoside triphosphates. The first primer may also be referenced as a polyT anchor primer in connection with reverse transcription or as a reverse primer in connection with PCR amplification. Then, a DNA molecule that includes the cDNA sequence is amplified by PCR in a reaction mixture that includes (i) the cDNA, (ii) the first primer that serves as the reverse primer; (iii) a second primer that serves as the forward primer; (iv) a DNA polymerase; and (v) all four deoxyribonucleoside triphosphates. The second or forward primer has sufficient complementarity to the 5′ nucleotides of the cDNA to hybridize with the cDNA and initiate synthesis of an extension product. Subsequently, the amplified DNA molecule is detected and/or quantified if present, wherein the presence and/or quantity of the amplified DNA corresponds to the presence and/or quantity, respectively, of the small RNA in the original sample. The mixture of components for RT and PCR may constitute one mixture for performing both the RT and PCR in a one-step RT-PCR method according to the present teachings.

In the methods described above, the RNA extension step of polyadenylation is performed using a poly(A) polymerase such as, for example, an E. coli poly(A) polymerase (E-PAP) such as E. coli poly(A) polymerase I (E-PAP I) or an E. coli poly(A) polymerase II (E-PAP II), a yeast poly(A) polymerase or any other suitable poly(A) polymerase. For illustrative purposes, but not as a limitation, polyadenylation may be carried out using from about 100 ng to about 1 μg total RNA in a volume of from about 20 to about 25 μL. The reaction mixture may contain buffer with optimized concentrations of MnCl₂, ATP and poly(A) polymerase and this may be incubated at about 37° C. for about 30 to about 60 min. The polyadenylated small RNA thus formed contains a sequence of 12 contiguous A residues. The sequence of contiguous A residues may be 12 or more A residues. Subsequent to the polyadenylation step, the polyadenylated RNAs may then be diluted with RNase-free water for PCR analysis or, in some embodiments, purification of the polyadenylated RNA may be performed prior to PCR, such as by phenol/chloroform extraction followed by ethanol precipitation or spin column.

Further, by way of illustration, but not as a limitation, one-step RT-PCR may be performed using the polyadenylated RNA solution, diluted by at least about 100 fold such that the original volume of polyadenylation reaction does not exceed 1% of the volume of the RT-PCR reaction mixture. Appropriately diluted polyadenylated RNA may then be added to the RT-PCR reaction mixture with the components of the RT-PCR reaction including buffer, reverse transcriptase, DNA polymerase and primers in a volume of from about 20 to about 25 μL.

The reverse transcriptase used in the one-step RT-PCR may be, for example, Moloney murine leukemia virus reverse transcriptase or Avian myeloblastosis virus reverse transcriptase or any other suitable reverse transcriptase. Further, the DNA polymerase may be, for example, a Taq DNA polymerase or a Tth DNA polymerase or a Tfl DNA polymerase or a Tli DNA polymerase or any other suitable thermostable DNA polymerase.

The first primer also referenced as the polyT anchor primer serves as both the primer for reverse transcription and the reverse primer for PCR amplification. In various embodiments, this primer contains not more than 40 nucleotides in length or not more than 35 nucleotides in length or not more than 30 nucleotides in length. The polyT anchor primer is specific for the particular small RNA being detected by virtue of having at least two 3′ terminal nucleotides that are complementary to at least two 3′ terminal nucleotides of the small RNA. The polyT anchor primer further contains a sequence of 12 contiguous T residues complementary to the sequence of contiguous A residues of the polyadenylated small RNA. The sequence of contiguous T residues may be 12 or more T residues. As a result, the polyT anchor primer hybridizes to the polyadenylated small RNA and synthesis of a cDNA complementary to the polyadenylated small RNA may be initiated. Further, the polyT anchor primer also serves as the reverse primer for PCR amplification.

Thus, the polyT anchor primer, which is also the PCR reverse primer, may have a sequence that may be represented from 5′ to 3′ as “ . . . xxxxxxxxTₙYY” in which “YY” selectively pairs with 3′ terminal nucleotides of the small RNA, “n” is the number of T residues and “xxxxxxxx . . . ” is an arbitrary sequence designed to improve the primer's melt temperature value. A simple design for the small RNA specific polyT anchor primer may include 2 selective bases as the 3′ terminal nucleotides of the primer. Thus, with this design, only 12 anchor primers would be needed for all small RNAs assuming the remaining portions of the sequence remain constant. The number, n, of the T residues may be 12 nucleotides or more for efficient annealing to the polyA tail of the small RNA. The remaining arbitrary residues added to 5′ end of the polyT anchor (reverse) primer may be selected to optimize or adjust the primer's melt temperature value. This may be helpful, at least in part, to achieve similar melt temperatures for the polyT anchor primer and the PCR forward primer. In one example of an implementation of this embodiment, the polyT anchor primer may have about 19 nucleotides. One particular polyT anchor primer that may be used is CGACTCACTATAGGGTTTTTTTTTTTTVN (SEQ ID NO:1), where V is A, G or C and N is A, T, G or C.

The second primer serves as the forward primer for PCR amplification. This primer is also specific for the particular small RNA by virtue of having a sequence of nucleotides complementary to the 3′ terminal end of the cDNA so as to hybridize therewith and initiate and initiate synthesis of an extension product. This primer is, thus, specific for the particular small RNA being detected and/or amplified by virtue of having a portion of its 3′ terminal sequence correspond to the 3′ terminal end of the small RNA and complementary to the 3′ terminal end of the cDNA. In some, but not all implementations, one or more LNA bases may be incorporated into the small RNA specific portion of the primer to increase specificity and improve the ability of the primer to discriminate the small RNA from closely related small RNA sequences, particularly during PCR amplification. In addition, the forward primer may have additional nucleotides at the 5′ end that serve to optimize or adjust its melt temperature. In an example of an implementation, the reverse primer may have about 19 nucleotides.

In various embodiments, the PCR amplification step may involve an amplicon of not more than about 80 nucleotides or an amplicon of not more than about 60 nucleotides.

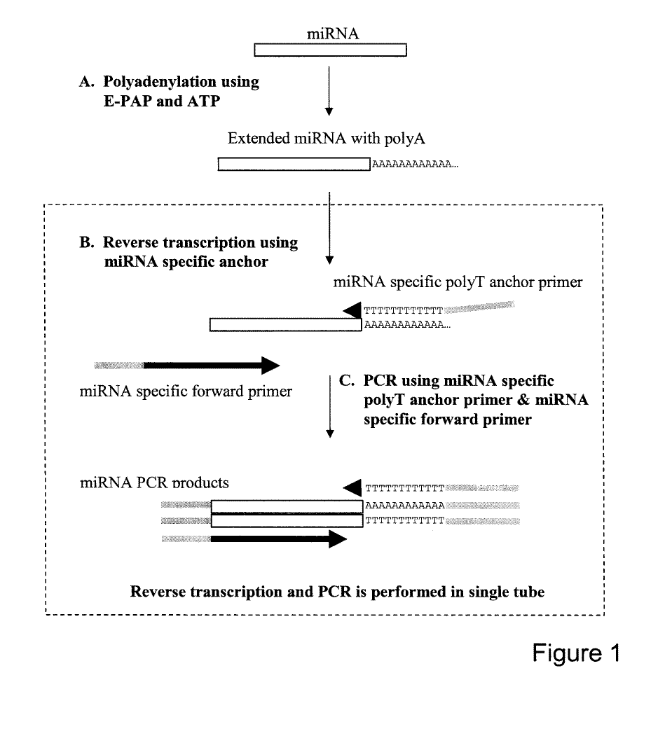

One example of an implementation of the method of the present teachings is illustrated in FIG. 1 in which the small RNA is represented by an miRNA. As shown in the figure, the method involves (A) polyadenylating the small RNA with an E. coli poly(A) polymerase (E-PAP) and ATP to form a polyadenylated small RNA; (B) reverse transcription using an miRNA specific poly(T) anchor primer and (C) PCR using the miRNA specific poly(T) anchor primer and an miRNA specific forward primer.

Detection and/or quantification may be performed by any of a number of approaches. One possible approach that may be used is real time RT-PCR. This approach is based upon Cycle Threshold (Ct), which is the fractional cycle number at which the PCR products are accumulated to a fixed threshold, which may be an arbitrary level. Accumulation of PCR products in the PCR process can be monitored in real time by fluorescence chemistry. One relatively simple and inexpensive approach may involve use of SYBR® green, which is a double strand DNA specific fluorescence dye. However, other fluorescence chemistries may also be used such as, for example, Sunrise primers, LUX fluorogenic primers (Invitrogen, Inc.) and the like (for review, see Wong & Medrano, 2005, Biotechniques 39:75-85).

Detection and/or quantification may also be performed using an end-point detection method. One such method may involve gel electrophoresis followed by evaluation of bands. Typically, a series of dilutions of a sample containing the small RNA are made for RT and PCR amplification which then may be used to generate of standard curve for the amount of PCR product in the various bands obtained in the gel electrophoresis. Semi-quantitative data may be obtained by staining the gel bands, for example, with ethidium bromide and evaluation of fluorescent intensity of the bands.

Included in the present teachings are methods that constitute portions of the method for detecting and/or quantifying a small RNA as described above. The present teachings thus provide a method of amplifying a polyadenylated small RNA that has a sequence of contiguous A at the 3′ terminal end. The sequence of contiguous A residues may be 12 or more A residues. The method includes reverse transcribing the polyadenylated small RNA to form a cDNA in a mixture that includes (i) the polyadenylated small RNA; (ii) a first primer of not more than 40 nucleotides in length having complementarity to at least two 3′ terminal nucleotides of the small RNA and the sequence of contiguous A residues of the polyadenylated small RNA so as to hybridize therewith and initiate synthesis of a cDNA complementary to the polyadenylated small RNA; (iii) a reverse transcriptase; and (iv) all four deoxyribonucleoside triphosphates. Subsequently, a DNA molecule that includes the cDNA sequence is amplified by PCR in a mixture that includes (i) the cDNA; (ii) the first primer; (iii) a second primer that is sufficiently complementary to the 3′ nucleotides of the cDNA to hybridize with the cDNA and initiate synthesis of an extension product; (iv) a DNA polymerase and (v) all four deoxyribonucleoside triphosphates. In some methods of the present teachings, the first primer may be of not more than 30 nucleotides in length. The mixture of components for RT and PCR may constitute one mixture for performing both the RT and PCR in a one-step RT-PCR method according to the present teachings. Thus, in various embodiments, both the reverse transcription and the PCR may be performed in a single tube and in the same mixture of reagents.

The present teachings also describe compositions that form a reaction mixture for performing an RT-PCR method on a polyadenylated small RNA. The reaction mixture includes (i) a sample containing a small RNA that has been polyadenylated to contain a sequence of contiguous A residues at the 3′ end, (ii) a first primer of not more than 40 nucleotides in length in length having complementarity to at least two 3′ terminal nucleotides of the small RNA and the sequence of contiguous A residues of the polyadenylated small RNA so as to hybridize to the polyadenylated small RNA and initiate synthesis of a cDNA complementary to the polyadenylated small RNA, (iii) a second primer that is complementary to the 3′ nucleotides of the cDNA so as to hybridize with the cDNA and initiate synthesis of an extension product; (iv) a reverse transcriptase, (v) a DNA polymerase and (vi) all four deoxyribonucleoside triphosphates. The sequence of contiguous A residues may be 12 or more A residues. In some of the reaction mixtures of the present teachings, the first primer and/or the second primer may have not more than 30 nucleotides in length.

In still other embodiments, the present teachings provide a method of amplifying a small RNA that has been polyadenylated to contain a sequence of contiguous A residues at the 3′ terminal end. The sequence of contiguous A residues may be 12 or more A residues. The method comprises (a) forming a first reaction complex comprising a first DNA primer of not more than 40 nucleotides in length, hybridized to a portion of the polyadenylated small RNA containing at least two nucleotides that formed the 3′ terminal end of the small RNA prior to polyadenylation and the sequence of contiguous A residues; (b) extending the first DNA primer to form an elongated cDNA molecule complementary to the polyadenylated small RNA; (c) separating the elongated cDNA molecule from the polyadenylated small RNA; (d) forming a second reaction complex comprising a second DNA primer hybridized to the 3′ end of the elongated cDNA molecule; (e) extending the second DNA primer to form a first strand; (f) separating the first strand from the elongated cDNA molecule; (g) forming a third reaction complex comprising the first strand and the first DNA primer; (h) extending the first DNA primer to form a second strand, wherein the first strand is hybridized to the second strand to form a double stranded complex; and (i) amplifying the double stranded complex. In some of the methods of the present teachings, the first primer and/or the second primer may have not more than 30 nucleotides in length.

In other embodiments, the present teachings provide a kit for detecting and/or quantifying the presence of a small RNA in a sample. In some, but not all of such embodiments, the kit may contain a poly(A) polymerase for converting the small RNA into a polyadenylated small RNA that will typically have a sequence of contiguous A residues at the 3′ terminal end. a sequence of contiguous A residues. The sequence of contiguous A residues may be 12 or more A residues. The poly(A) polymerase may be, for example, an E. coli poly(A) polymerase (E-PAP) such as E. coli poly(A) polymerase I (E-PAP I) or an E. coli poly(A) polymerase II(E-PAP II), a yeast poly(A) polymerase or any other suitable poly(A) polymerase. Further components of the kit may be designed to perform RT-PCR on the polyadenylated small RNA as described below. In other embodiments, the kit may be designed to perform RT-PCR on a small RNA molecule that is polyadenylated at the 3′ end. The polyadenylated form of the small RNA may have a sequence of contiguous A residues at the 3′ terminal end. The sequence of contiguous A residues may be 12 or more A residues.

In various embodiments, the kits may include components to perform RT-PCR on a polyadenylated form of the small RNA. Thus, included in the kits are a primer set comprising a first primer of not more than 40 nucleotides in length having complementarity to at least two 3′ terminal nucleotides of the small RNA and the sequence of contiguous A residues of the polyadenylated small RNA. The primer set further comprises a second primer that is sufficiently complementary to the 3′ nucleotides of the cDNA to hybridize with the cDNA and initiate synthesis of an extension product. The kits may also contain a reverse transcriptase and a DNA polymerase. In some implementations, the kits may also include a poly(A) polymerase for generating the polyadenylated form of the small RNA. In addition, the kits may include suitable dyes, reagents and buffers for performing the RT-PCR methods of the present teachings. The components of the kits may be packaged in a container.

The RT-PCR methods, compositions and kits according to the present teachings, may be used in any application for the detection and/or quantification of small RNAs. In one non-limiting area of application, the detection and/or quantification of certain small RNAs that may be potential biomarkers for cancer related processes might be useful in the diagnosis and treatment of the cancer (for review See Esquela-Kerscher & Slack, 2006, Nat. Rev. Cancer 6:259-269; Hammond, 2006, Curr. Opin. Genet. Dev. 16:4-9).

The following Examples further illustrate the invention and are not intended to limit the scope of the invention.

### Example 1

This example illustrates the amplification of small RNAs in one-step real time RT-PCR.

Total RNA of Human hela cells and Arabidopsis was purified by Trizol reagent (Invitrogen, Inc., Carlsbad, Calif.) according to the manufacturer's instructions. Before performing RT-PCR, total RNA of hela cells or Arabidopsis were polyadenylated by E-PAP I and ATP using RNA poly A tailed kit (Ambion, Inc., Austin, Tex.). Briefly, 1 μg total RNA was used in 20 μL reaction mixtures containing 4 μL 5×E-PAP I buffer, 2 μL of 25 mM McCl₂, 2 μL of 10 mM ATP and 1 μL of E-PAP I at a concentration of 2 U/μL. Reaction mixtures were then incubated at 37° C. for 1 hour. After polyadenylation, the mixtures were diluted with RNase-free water and used as templates for RT-PCR.

The small RNAs evaluated by the present method were an miRNA from human cells and an miRNA from a plant of an Arabidopsis species and a human small non-coding RNA, small nuclear RNA U6, which is frequently used as internal reference standard. The small RNAs and primer sets were as follows.

Hsa-mir-21:

(SEQ ID NO:2)

5′-UAGCUUAUCAGACUGAUGUUGA-3′
Forward specific primer:

(SEQ ID NO:3)

5′-aaaaaaaaTAGCTTATCAGACTGATGT-3′
polyT specific anchor primer:

(SEQ ID NO:4)

5′-CGACTCACTATAGGGttttttttttttCA-3′
Ath-MIR167d:

(SEQ ID NO:5)

5′-UGAAGCUGCCAGCAUGAUCUGG-3′
Forward specific primer:

(SEQ ID NO:6)

5′-aaaaaaaaaTGAAGCTGCCAGCATGAT-3′
polyT specific anchor primer:

(SEQ ID NO:7)

5′-CGACTCACTATAGGGttttttttttttCC-3′
Human U6 RNA (H. sapiens, X07425):

(SEQ ID NO:8)

5′-CTGCGCAAGGATGACACGCAAATTCGTGAAGCGTTCCATATTT
TT-3′
Forward specific primer:

(SEQ ID NO:9)

5′-CTGCGCAAGGATGACACGCA-3′
polyT specific anchor primer:

(SEQ ID NO:10)

5′-CGACTCACTATAGGGttttttttttttAA-3′

Real-time RT-PCR was performed using a SuperScript™ III PLATINUM SYBR® Green One-Step qRT-PCR kit (Invitrogen, Inc., Carlsbad, Calif.) on an Applied Biosystems 7900HT Sequence Detection System (Applied Biosystems, Inc., Foster City, Calif.). The 20 μL PCR reaction mixture included diluted polyadenylated RNA, which amounted to 100 pg original total RNA, 1×SYBR® Green Reaction Mix, 0.5 μM forward primer and 0.5 μM reverse primer, 0.4 μL ROX reference Dye. The reaction mixtures were incubated in 96 well plates at 42° C. for 5 min and then 95° C. for 10 min followed by 40-50 cycles of 95° C. for 15 s, and 60° C. for 1 min. This was followed by thermal denaturation to generate dissociation curves.

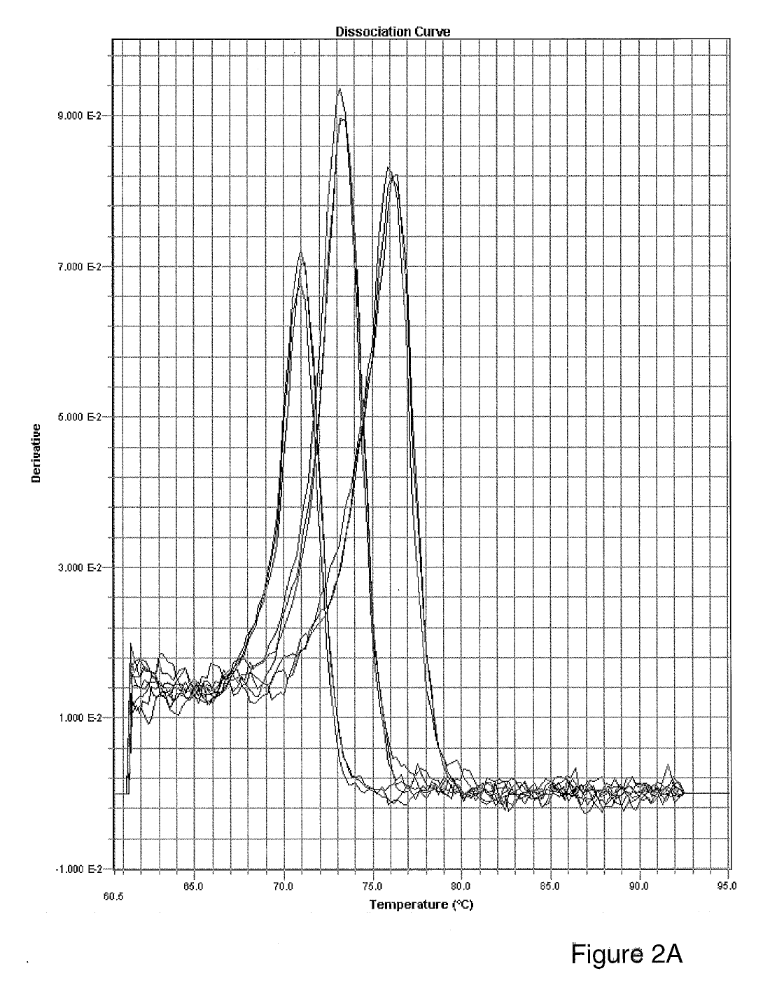

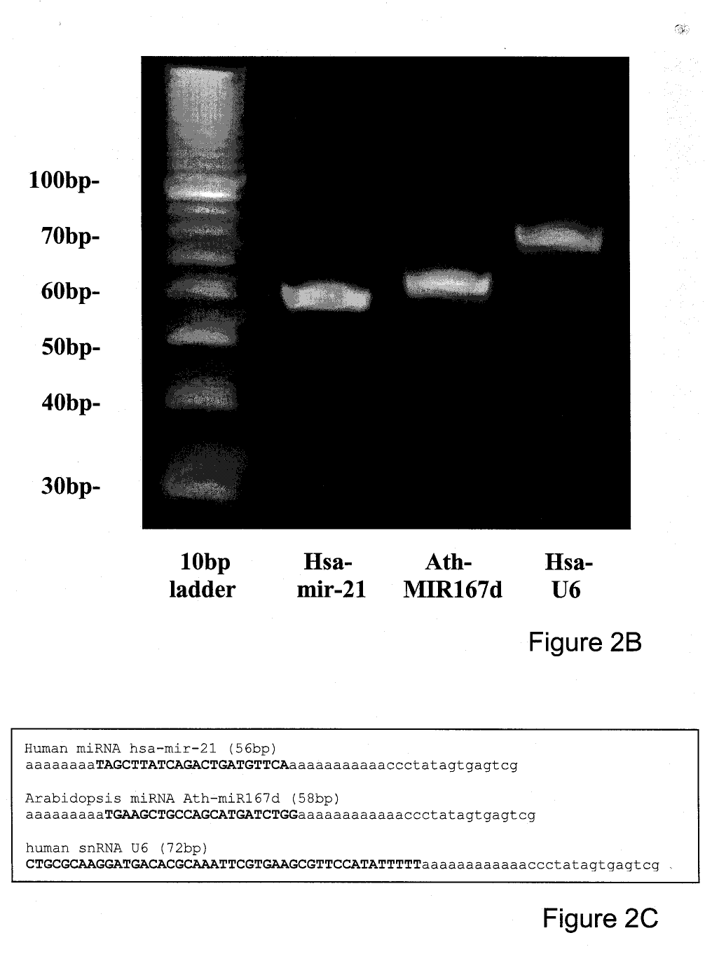

Results show that all detected miRNAs and U6 RNA were amplified based upon dissociation curve analysis of final PCR products (FIG. 2A) and gel electrophoresis (FIG. 2B). Furthermore, the Tₘ values estimated from the dissociation curve analysis and the sizes of PCR products revealed from gel electrophoresis were as predicted from the sequences (FIG. 2C). These results validated the RNA extension-based one-step RT-PCR method for amplification of small RNAs.

### Example 2

This example illustrates amplification efficiency, sensitivity and dynamic range of the one-step RT-PCR method for small RNAs.

Ten-fold dilution series of synthetic RNA oligonucleotides or total RNA were used as the template for real-time PCR to generate plots of log copy numbers of the tested miRNA at different dilutions versus the corresponding threshold cycle (Ct). Amplification efficiency of the real-time PCR was determined as follows. The slope of the linear plot was defined as −(1/log E), where E is the amplification efficiency. The value for E should approach 2 as the efficiency reaches the maximum (Livak and Schmittgen, 2001, Methods 25:402-408)

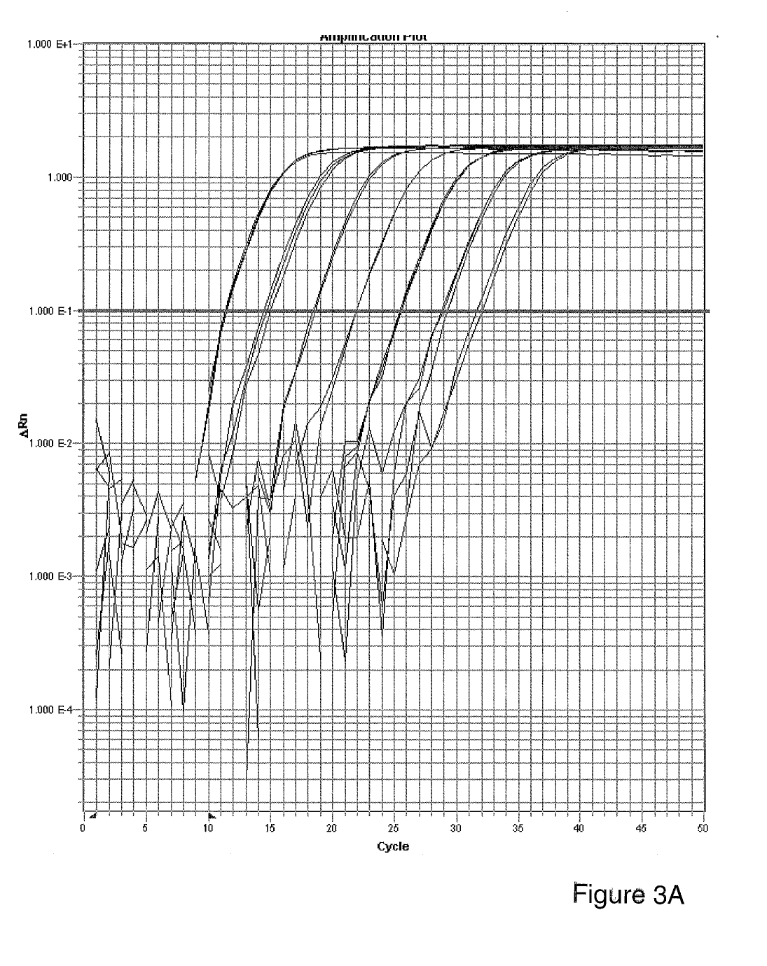

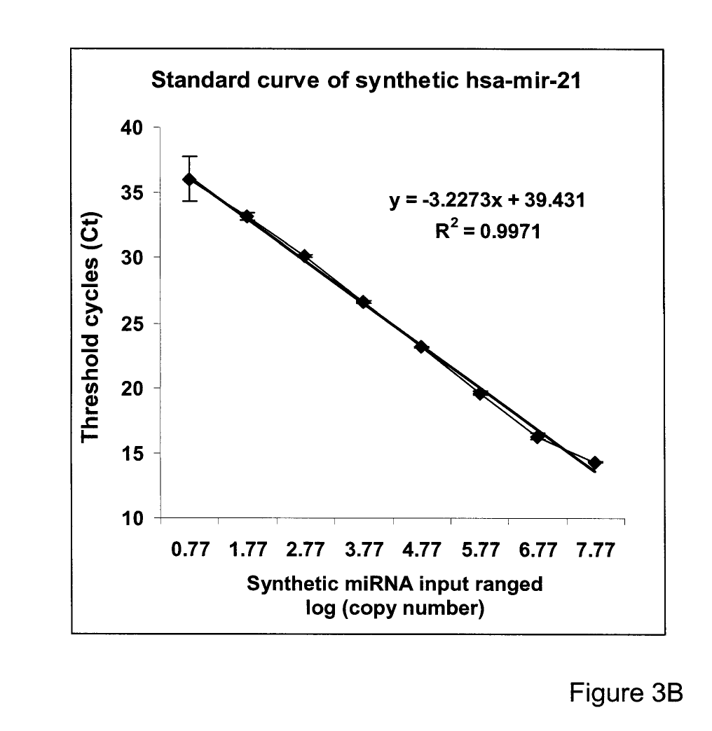

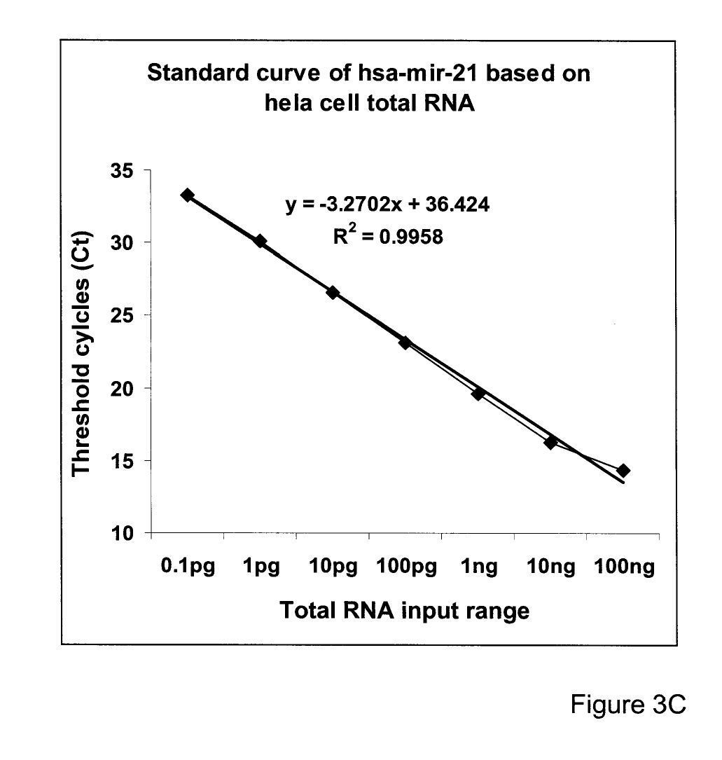

In experiments using synthetic RNA oligonucleotides, 1 pmol synthetic RNA oligonucleotide of the human miRNA hsa-mir-21, was added to 1 μg Arabidopsis total RNA for polyadenylation and then diluted with RNase-free water in a series of reaction mixtures representing several orders of magnitude. The results shown in FIGS. 3A and B show that one-step real time RT-PCR using these diluted samples produced very good linearity between the log of target input and Ct value for hsa-mir-21 amplification. The results as shown in FIG. 3 further demonstrate that this RT-PCR assay has a wide dynamic range of at least 6 log units, which ranged from 6×10⁷ to 6 copies. The assay is also very sensitive, inasmuch as it was able to quantitatively detect as few as 6 copies of miRNA in the reaction mixtures. Amplification efficiency for the synthetic RNA oligonucleotides approached 2, which the ideal value for PCR amplification (see FIGS. 3A and B)

In evaluating experiments using total RNA, 10 μg total RNA purified from human Hela cells was polyadenylated in 100 μL polyadenylation reaction mixture containing the same amounts and concentrations of buffer, MnCl₂, ATP and E-PAP I as described in Example 1. Purification was then performed by phenol/chloroform extraction followed by ethanol precipitation. Purified polyadenylated RNA was then dissolved in RNase-free water and diluted into a series for PCR analysis for hsa-mir-21. The results in FIG. 3 show that a wide linear range of 6 logs was obtained for hsa-mir-21 detection, which amounts to 0.1 pg to about 100 ng of original RNA per reaction mixture. In addition, amplification efficiency approached 2.

### Example 3

This example illustrates the specificity of real-time RT-PCR assay for small RNAs using mismatched primers.

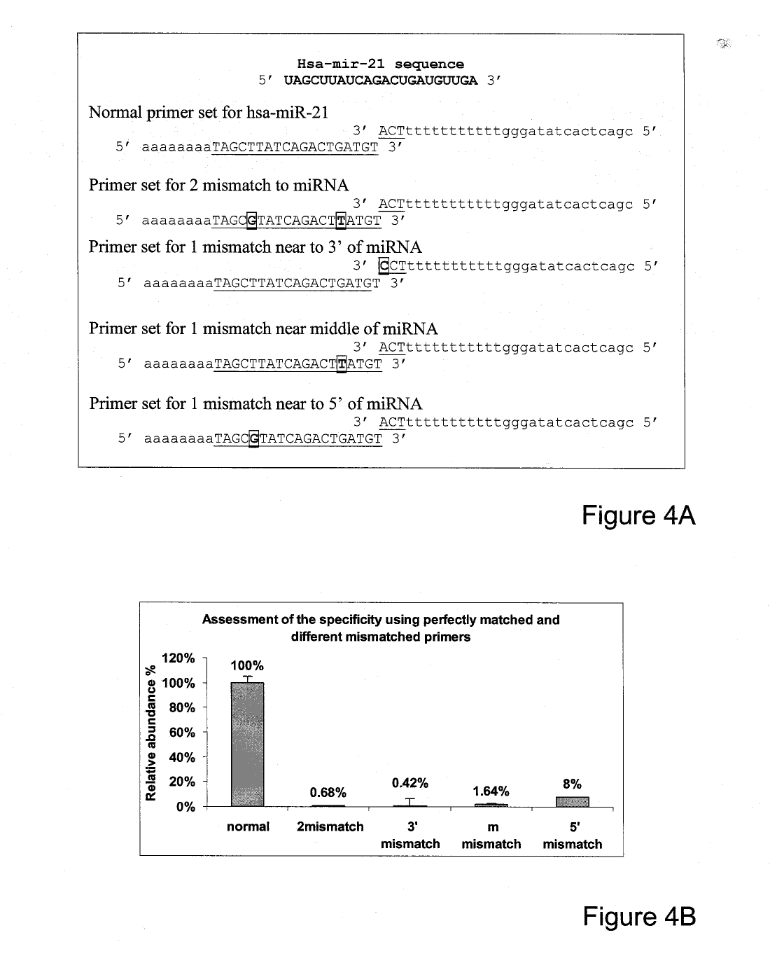

Assay specificity was tested by using primer sequences with mismatched nucleotides as shown in FIG. 4A. Small RNAs, in amounts corresponding to 100 pg total RNA, were used as templates for one-step real time RT-PCR with primer sets shown in FIG. 4A.

Quantification data for the different primer sets was based upon the assumption that amplification was 2 for all primer sets. Calculations were performed using the formula 2^{-ΔCt}, where ΔCt=(Ct_{test primer set}−Ct_{normal primer set}). Because PCR using the normal primer set should generate the lowest Ct value, the Ct value for the normal primer set was assumed to represent 100% and its Ct value was used for normalization in each comparison. The results show that no PCR amplification occurred using a primer with 2 mismatched nucleotides, whereas primers with 1 mismatch showed greatly reduced amplification compared to amplification with the normal primer even where the one mismatch was located in a position that corresponds to a position near the 5′ terminal end of the miRNA sequence (see FIG. 4B).

### Example 4

This example illustrates the effects of miRNA precursor (pre-miRNA) on RT-PCR and quantification of miRNA.

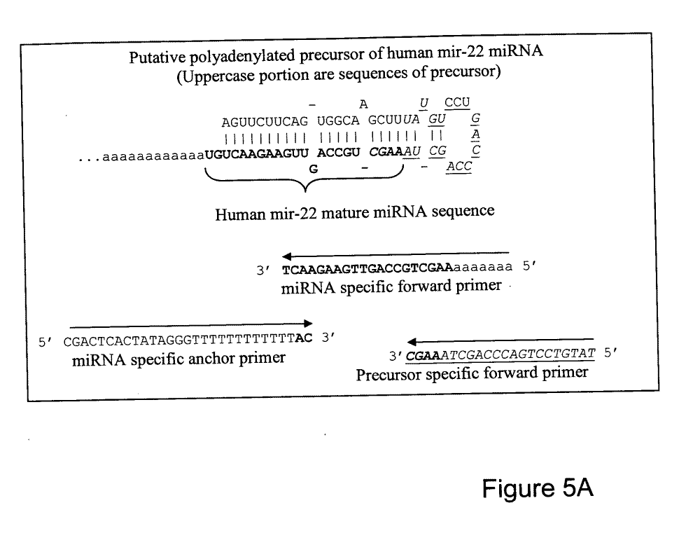

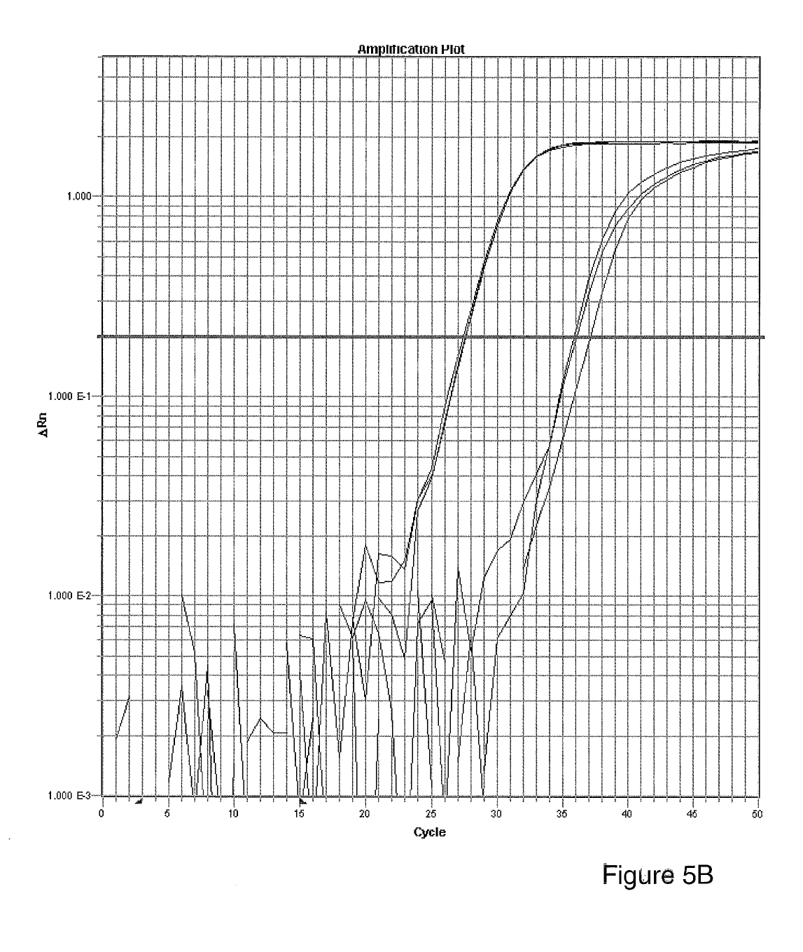

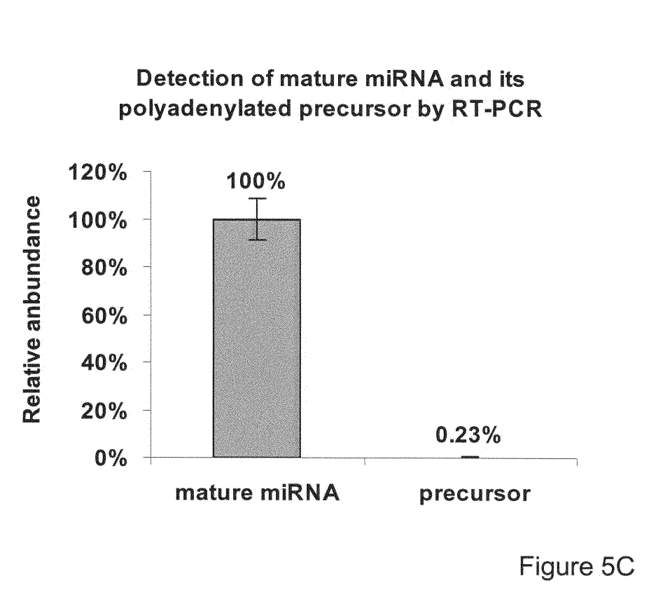

This example tested the possibility that the amplified PCR products of the miRNA specific polyT anchor primer and the miRNA specific forward primer might include sequences derived from pre-miRNA, which is a spliced intermediate product of RNase III enzyme generated from the original transcript of the miRNA gene. The sequence of human mir-22 miRNA precursor (pre-miRNA) is shown in FIG. 5A. Furthermore, the amplification products derived from the pre-miRNA are the same size as that of the mature miRNA so that it is relevant to determine whether amplification of pre-miRNA is likely to effect the measurement of values for the mature miRNA.

The human miRNA, hsa-mir-22 and its pre-miRNA were analyzed. The miRNA mature sequence is located in the 3′arm of its pre-miRNA and there is only one possible pre-miRNA for this miRNA. The abundance of polyadenylated pre-miRNA was quantified in the RNA extension based one-step RT-PCR using a pre-miRNA specific forward primer and the miRNA specific reverse primer and this was compared to that obtained for the mature miRNA using mature miRNA specific primers as shown in FIG. 5A. Results in FIGS. 5B and C show that the amplification of the polyadenylated pre-miRNA is far less than that of the mature miRNA. Based upon the difference of Ct values for the pre-miRNA and the mature miRNA, the relative abundance of the pre-miRNA is less than 0.3% of that of the mature miRNA. This value of 0.3% is far less than the variation of duplicated reactions using real time PCR which is usually considered to be about 5%. Thus, the small amount of precursor can be ignored in the quantification of miRNA in the one-step RT-PCR assay.

### Example 5

This example illustrates the use of one-step RT-PCR for quantifying tissue-specific expression of various miRNAs in human and plant tissues.

Quantification of miRNAs was performed by real time RT-PCR and by conventional RT-PCR with end-point detection. Both approaches were then validated by Northern hybridization.

The three approaches evaluated miRNA from various human tissues and miRNA from various plant tissues. The human miRNA tissues evaluated were brain (Br), heart (He), kidney (Ki), liver (Li), lung (Lu), skeleton muscle (Sk), spleen (Sp) and thymus (Th). Expression levels were determined in these tissues for the miRNAs, hsa-miR-21 and hsa-miR-122. The plant tissues evaluated were leaf (L), phloem (P), shoot tip (S) and xylem (X) tissues from P. trichocarpa. Expression levels were determined for the miRNAs, ptc-miR408, ptc-MIR166 and ptc-MIR167. The miRNA, ptc-miR408 belongs to a single MIR gene family, whereas the ptc-MIR166 gene family has 20 members which encode two groups of mature miRNA sequences. The ptc-miR166a-m miRNAs represent type 1 sequences (seq. 1) and the ptc-mirR166n˜p miRNAs represent type 2 sequences (seq. 2). The ptc-MIR167 miRNA family encodes three possible groups of miRNAs, with ptc-miR167a˜d having type 1 sequences (seq. 1), ptc-miR167e and h having type 2 sequences (seq. 2) and ptc-miR167f and g having type 3 sequences (seq. 3).

Total RNAs of different human tissues were purchased from Ambion (Austin, Tex.). Total RNAs of different tissues of P. trichocarpa's were purified by Plant RNA reagent (Invitrogen, Inc., Carlsbad, Calif.) according to the manufacturer's instructions. Before performing RT-PCR, total RNAs were polyadenylated by E-PAP I and ATP using RNA poly A tailed kit (Ambion, Inc., Austin, Tex.). Briefly, 0.1 μg total RNA was used in 5 μL reaction mixtures containing 1 μL 5×E-PAP I buffer, 0.5 μL of 25 mM MCCl₂, 0.5 μL of 10 mM ATP and 0.2 μL of E-PAP I. Reaction mixtures were then incubated at 37° C. for 1 hour. After polyadenylation, the mixtures were diluted with RNase-free water and used as templates for RT-PCR.

For quantification of these miRNAs, the following primer sets for miRNAs, reference 5S rRNA and 5.8S rRNA were used.

hsa-miR-21:

(SEQ ID NO:2)

5′ UAGCUUAUCAGACUGAUGUUGA 3′
Forward primer:

(SEQ ID NO:3)

5′ aaaaaaaaTAGCTTATCAGACTGATGT 3′
Reverse primer:

(SEQ ID NO:4)

5′ cgactcactatagggttttttttttttCA 3′
hsa-miR-122:

(SEQ ID NO:11)

5′ UGGAGUGUGACAAUGGUGUUUG 3′
Forward primer:

(SEQ ID NO:12)

5′ aaaaaaaaTGGAGTGTGACAATGGTGTT 3′
Reverse primer:

(SEQ ID NO:4)

5′ cgactcactatagggttttttttttttCA 3′
Human 5S:

(SEQ ID NO:13)

5 GGAATACCGGGTGCTGTAGGCTTT 3′
Forward primer:

(SEQ ID NO:14)

5′ aaaaaaaaaGGAATACCGGGTGCTGTAG 3′
polyT anchor primer:

(SEQ ID NO:10)

5′ cgactcactatagggttttttttttttAA 3′
ptc-miR-408:

(SEQ ID NO:15)

5′ UAGCUUAUCAGACUGAUGUUGA 3′
Forward primer:

(SEQ ID NO:16)

5′ aaaaaaaaaTGCACTGCCTCTTCCCTGG 3′
Reverse primer:

(SEQ ID NO:17)

5′ cgactcactatagggttttttttttttGC 3′
ptc-miR-166a~m:

(SEQ ID NO:18)

5′ UCGGACCAGGCUUCAUUCCCC 3′
Forward primer:

(SEQ ID NO:19)

5′ aaaaaaaaTCGGACCAGGCTTCATTC 3′
Reverse primer:

(SEQ ID NO:20)

5′ cgactcactatagggttttttttttttGG 3′
ptc-miR166n~q:

(SEQ ID NO:21)

5′ UCGGACCAGGCUUCAUUCCUU 3′
Forward primer:

(SEQ ID NO:22)

5′ aaaaaaaaTCGGACCAGGCTTCATTC 3′
Reverse primer:

(SEQ ID NO:10)

5′ cgactcactatagggttttttttttttAA 3′
ptc-miRl67a~d:

(SEQ ID NO:23)

5′ UGAAGCUGCCAGCAUGAUCUA 3′
Forward primer:

(SEQ ID NO:24)

5′ aaaaaaaaaTGAAGCTGCCAGCATGAT 3′
Reverse primer:

(SEQ ID NO:25)

5′ cgactcactatagggttttttttttttAG 3′
ptc-miR167f,g:

(SEQ ID NO:26)

5′ UGAAGCUGCCAGCAUGAUCUU 3′
Forward primer:

(SEQ ID NO:27)

5′ aaaaaaaaaTGAAGCTGCCAGCATGAT 3′
Reverse primer:

(SEQ ID NO:10)

5′ cgactcactatagggttttttttttttAA 3′
ptc-miR167e,h:

(SEQ ID NO:28)

5′ UGAAGCUGCCAGCAUGAUCUG 3′
Forward primer:

(SEQ ID NO:29)

5′ aaaaaaaaaTGAAGCTGCCAGCATGAT 3′
Reverse primer:

(SEQ ID NO:4)

5′ cgactcactatagggttttttttttttCA 3′
ptc-5.8S rRNA:

(SEQ ID NO:30)

5′ GGCACGUCUGCCUGGGUGUCACGC 3′
Forward primer:

(SEQ ID NO:31)

5′ aaaaaaaaaCGTCTGCCTGGGTGTCAC 3′
Reverse primer:

(SEQ ID NO:17)

5′ cgactcactatagggttttttttttttGC 3′

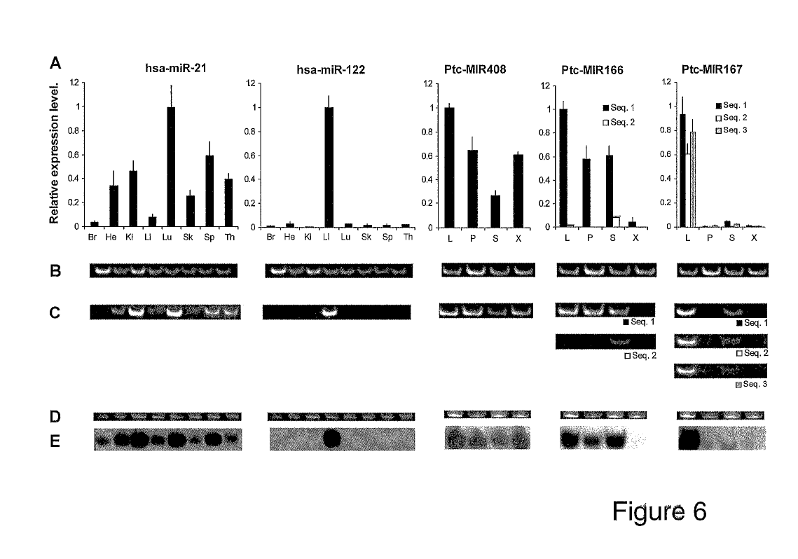

Quantification of miRNA by real-time RT-PCR was performed as described in Example 1, using template polyA tailed RNAs amounts of 100 μg original total RNA for each RT-PCR reaction. Expression values for human miRNA were normalized by human 5S rRNA and the values for P. trichocarpa populus' miRNA (ptc-miRNA) were normalized by its 5.8S rRNA (ptc-5.8S). Results from one-step real time RT-PCR on various human tissues and tissues from P. trichocarpa are shown in FIG. 6A.

For end-point quantification of miRNAs, conventional RT-PCR reaction mixtures were prepared in the same manner as real time RT-PCR reaction mixtures as described above. The RT-PCRs were then performed for hsa-miR-21 (25 cycles), hsa-miR-122 (30 cycles), ptc-miR408 (30 cycles), members of ptc-MIR166 (30 cycles) and ptc-MIR167 (30 cycles) and reference RNAs, human 5S rRNA (25 cycles) and P. trichocarpa 5.8S rRNA (20 cycles) for the various tissues. For end-point quantification of the miRNAs, RT-PCR products of different PCR cycles were detected and analyzed by 5% non-denatured polyacrylamide gel electrophoresis followed by staining with ethidium bromide. Relative quantification of the tested miRNAs was estimated by signal strength of PCR products (band) on gel compared to that obtained for reference rRNAs (See FIGS. 6B and C).

For validation of RT-PCR results, Northern hybridization analyses using ³²P-end-labeled anti-sense DNA oligos for hsa-miR-21, hsa-mir-122, ptc-miR408, ptc-miR166a˜m and ptc-miR-167a˜g for analyzed miRNAs or miRNA families were performed. Probes sequences complementary to ptc-miR166a were used for the ptc-MIR166 miRNAs and probe sequences complementary to ptc-miR167a were used for the ptc-MIR167 miRNAs.

For Northern hybridization analyses, 10 μg total RNA of samples to be tested were separated on 12% polyacrylamide/8M urea gel (Amersham Pharmacia, Uppsala, Sweden) in a Protean II apparatus (BioRad, Hercules, Calif.). Loading controls consisted of reference rRNA transcripts separated on the polyacrylamide/urea gels and stained with ethidium bromide (FIG. 6D).

For analysis of miRNAs, the gels were electro-blotted onto Hybond-N⁺ membrane (Amersham Biosciences, Piscataway, N.J.) by Trans-Blot SD semi-dry electrophoretic transfer cell (BioRad, Hercules, Calif.). After UV cross-linking and air drying, blotted membranes were prehybridized with Ultrahyb-oligo hybridization buffer (Ambion, Inc., Austin, Tex.) at 40° C. for 60 min, then hybridized with ³²P-end-labeled antisense probes for the tested miRNAs or miRNA families prepared by T4 polynucleic kinase (Fisher Scientific, Fairlawn, N.J.), and incubated at 37° C. overnight. The membranes were washed twice at 37° C. with 2×SSC and 0.5% SDS for 15 min and exposed to an X-ray film (Kodak, Rochester, N.Y.) at −80° C. for signal visualization. For reuse, the membranes were stripped by boiling in 0.1% SDS, cooled to room temperature, and washed once with 2×SSC.

Results in FIG. 6E show that the amounts of miRNA obtained with Northern hybridization analyses were comparable to that obtained with one-step real-time RT-PCR (FIG. 6A) and end-point RT-PCR (FIG. 6C).

Overall results show that the polyA tailing based one-step RT-PCR method provided an accurate real time detection method that yielded results comparable to RT-PCR with end-point detection. Furthermore, both approaches were validated by classic Northern hybridization analysis.

The descriptions in this application are exemplary and explanatory only and are not intended to limit the scope of the current teachings. Further, the use of the singular includes the plural unless specifically stated otherwise. Also, the term “and/or” is intended to mean that the terms before and after can be taken together or separately and the expression in which it is used, as illustrated by “X and/or Y”, is intended to be synonymous with the expression “either or both of X and Y”.

All literature references and similar materials cited in this application, including, patents, patent applications, articles, books, treatises, and internet web pages are expressly incorporated by reference in their entirety for any purpose. In the event that one or more of the incorporated literature references uses a term in such a way that it contradicts that term's definition in this application or makes a statement that contradicts or is inconsistent with the teachings in this application, this application and its teachings are controlling.

What is claimed is:

1. A method for detecting and/or quantifying a small RNA, the method comprising:
(a) polyadenylating the small RNA with ATP and a poly(A) polymerase to form a polyadenylated small RNA having a sequence of contiguous A residues;
(b) reverse transcribing the polyadenylated small RNA to form a cDNA in a reaction mixture comprising (i) a first primer of not more than 40 nucleotides in length having complementarity to at least two 3′ terminal nucleotides of the small RNA and the sequence of contiguous A residues of the polyadenylated small RNA so as to hybridize therewith and initiate synthesis of a cDNA complementary to the polyadenylated small RNA, (ii) a reverse transcriptase and (iii) all four deoxyribonucleoside triphosphates;
(c) amplifying a DNA molecule comprising the cDNA in a reaction mixture comprising (i) the cDNA, (ii) the first primer; (iii) a second primer that is sufficiently complementary to the 3′ nucleotides of the cDNA to hybridize therewith and initiate synthesis of an extension product; (iv) a DNA polymerase and (v) all four deoxyribonucleoside triphosphates; and
(d) detecting and/or quantifying the amplified DNA molecule, wherein the presence and/or quantity of the amplified DNA corresponds to that of the small RNA.

2. The method of claim 1, wherein the sequence of contiguous A residues is a sequence of 12 or more A residues.

3. The method of claim 1, wherein converting the polyadenylated small RNA to a cDNA and amplifying a DNA molecule comprising the cDNA are performed in a single tube and wherein converting the polyadenylated small RNA to a cDNA and amplifying a DNA molecule comprising the cDNA comprises one-step RT-PCR.

4. The method of claim 1, wherein detecting and/or quantifying the amplified cDNA molecule comprises utilizing real time RT-PCR.

5. The method of claim 1, wherein the first primer comprises from 5′ to 3′, an arbitrary sequence of about 15 nucleotides, about 12 contiguous T residues and two nucleotides complementary to 3′ terminal nucleotides of the small RNA.

6. The method of claim 5, wherein the first primer is CGACTCACTATAGGGTTTTTTTTTTTTVN (SEQ ID NO:1).

7. The method of claim 1, wherein the second primer comprises from 5′ to 3′, about 9 contiguous A residues and about 18 nucleotides complementary to 3′ terminal nucleotides of the cDNA.

8. The method of claim 1, wherein the small RNA is an miRNA, a siRNA′, an rasiRNA or a piRNA.

9. The method of claim 1, wherein detecting and/or quantifying the amplified cDNA comprises utilizing gel electrophoresis.

10. The method of claim 1, wherein the DNA polymerase is a Taq DNA polymerase, the reverse transcriptase is a Moloney Murine Leukemia Virus Reverse Transcriptase and the poly(A)polymerase is E. coli Poly(A) Polymerase I.

11. The method of claim 1, wherein amplifying the DNA molecule produces an amplicon having not more than 80 nucleotides.

12. A kit for detecting and quantifying the presence of a small RNA, the kit comprising a primer set comprising a (a) first primer of not more than 40 nucleotides in length having (i) at least two contiguous nucleotides complementary to the 3′ terminal end of the small RNA and (ii) a sequence of contiguous T residues 3′ to the at least two contiguous nucleotides so as to hybridizes to a 3′ polyadenylated form of the small RNA and initiate synthesis of an extension product; and (b) a second primer that is sufficiently complementary to the 3′ nucleotides of the cDNA to hybridize with the cDNA and initiate synthesis of an extension product, packaged in a container.

13. The kit of claim 12, further comprising a reverse transcriptase and a DNA polymerase.

14. The kit of claim 13 further comprising a poly(A) polymerase for generating the polyadenylated form of the small RNA.

15. The kit of claim 13, wherein the first primer comprises from 5′ to 3′, an arbitrary sequence of about 15 nucleotides, about 12 contiguous T residues and two nucleotides complementary to the at least two 3′ terminal nucleotides of the small RNA.

16. The kit of claim 13, wherein the second primer comprises from 5′ to 3′, about 9 contiguous A residues and about 18 nucleotides complementary to 3′ terminal nucleotides of the cDNA.

17. A method of amplifying a small RNA that has been polyadenylated to contain a sequence of contiguous, 3′-terminal A residues, the method comprising:
(a) reverse transcribing the polyadenylated small RNA to form a cDNA in a reaction mixture comprising (i) a first primer of not more than 40 nucleotides in length having complementarity to at least two 3′ terminal nucleotides of the small RNA prior to polyadenylation and the sequence of contiguous A residues of the polyadenylated small RNA so as to hybridize therewith and initiate synthesis of a cDNA complementary to the polyadenylated small RNA, (ii) a reverse transcriptase and (iii) all four deoxyribonucleoside triphosphates; and
(b) amplifying a DNA molecule comprising the cDNA by a polymerase chain reaction in a reaction mixture comprising (i) the cDNA, (ii) the first primer; (iii) a second primer that is sufficiently complementary to the 3′ nucleotides of the cDNA to hybridize with the cDNA and initiate synthesis of an extension product; and (iv) a DNA polymerase and (v) all four deoxyribonucleoside triphosphates.

18. The method of claim 17, wherein the first primer comprises from 5′ to 3′, an arbitrary sequence of about 15 nucleotides, about 12 contiguous T residues and two nucleotides complementary to 3′ terminal nucleotides of the small RNA prior to polyadenylation.

19. The method of claim 17, wherein the second primer comprises from 5′ to 3′, about 9 contiguous A residues and about 18 nucleotides complementary to 3′ terminal nucleotides of the cDNA.

20. A reaction mixture comprising (a) a sample containing a small RNA that has been polyadenylated to contain a sequence of contiguous A residues at the 3′ end; (b) a first primer of not more than 40 nucleotides in length having complementarity to at least two 3′ terminal nucleotides of the small RNA and the sequence of contiguous A residues of the polyadenylated small RNA so as to hybridize to the polyadenylated small RNA and initiate synthesis of a cDNA complementary to the polyadenylated small RNA, (c) a second primer that is complementary to the 3′ nucleotides of the cDNA so as to hybridize with the cDNA and initiate synthesis of an extension product, (d) a reverse transcriptase, (e) a DNA polymerase and (f) all four deoxyribonucleoside triphosphates.

21. The reaction mixture of claim 20, wherein the first primer comprises from 5′ to 3′, an arbitrary sequence of about 15 nucleotides, about 12 contiguous T residues and two nucleotides complementary to 3′ terminal nucleotides of the small RNA.

22. The reaction mixture of claim 20, wherein the second primer comprises from 5′ to 3′, about 9 contiguous A residues and about 18 nucleotides complementary to 3′ terminal nucleotides of the cDNA.

23. A method of amplifying a small RNA that has been polyadenylated to containing a sequence of contiguous A residues at the 3′ terminal end, the method comprising:
a) forming a first reaction complex comprising a first DNA primer of not more than 40 nucleotides in length, hybridized to a portion of the polyadenylated RNA containing at least two nucleotides that formed the 3′ terminal end of the small RNA prior to polyadenylation and the sequence of contiguous A residues;
b) extending the first DNA primer to form an elongated cDNA molecule complementary to the polyadenylated small RNA;
c) separating the elongated cDNA molecule from the polyadenylated small RNA;
d) forming a second reaction complex comprising a second DNA primer hybridized to the 3′ end of the elongated cDNA molecule;
e) extending the second DNA primer to form a first strand;
f) separating the first strand from the elongated cDNA molecule;
g) forming a third reaction complex comprising the first strand hybridized to the first DNA primer;
h) extending the first DNA primer to form a second strand, wherein the first strand is hybridized to the second strand to form a double stranded complex; and
i) amplifying the double stranded complex.

24. The method of claim 23, wherein the first primer comprises from 5′ to 3′, an arbitrary sequence of about 15 nucleotides, about 12 contiguous T residues and two nucleotides complementary to 3′ terminal nucleotides of the small RNA.

25. The method of claim 22, wherein the second primer comprises from 5′ to 3′, about 9 contiguous A residues and about 18 nucleotides complementary to 3′ terminal nucleotides of the cDNA.
